(19) United States
(12) Patent Application Publication
(10) Pub. No.: US 2012/0004508 A1
(43) Pub. Date: Jan. 5, 2012

(54) SURGICAL ILLUMINATOR WITH DUAL SPECTRUM FLUORESCENCE

(76) Inventors: Ian McDowall, Woodside, CA (US); Christopher J. Hasser, Los Altos, CA (US); Simon P. DiMaio, Sunnyvale, CA (US)

(21) Appl. No.: 12/855,905

(22) Filed: Aug. 13, 2010

Related U.S. Application Data
(60) Provisional application No. 61/361,220, filed on Jul. 2, 2010.

(51) Int. Cl. A61B 1/06

(52) U.S. Cl. 600/178

(57) ABSTRACT

In a minimally invasive surgical system, an illuminator includes a visible color component illumination source and a hardware non-visible fluorescence emission illumination source. Thus, the illuminator outputs target image illumination light in a first spectrum where the first spectrum includes at least a portion of the visible spectrum. The illuminator also outputs target image illumination light in a second spectrum, where the second spectrum includes non-visible light with a wavelength the same as a wavelength in an emission from a fluorophore.

### RELATED APPLICATION

This application claims priority to and the benefit of:

U.S. Provisional Application No. 61/361,220 filed Jul. 2, 2010 entitled “DUAL SPECTRUM SURGICAL ILLUMINATOR,” naming as inventors, Ian McDowall, Christopher J. Hasser, and Simon P. DiMaio, which is incorporated herein by reference in its entirety.

### BACKGROUND

1. Field of Invention

Aspects of this invention are related to endoscopic imaging and are more particularly related to generating fluorescence images without using fluorophores.

2. Related Art

The da Vinci® Surgical System, commercialized by Intuitive Surgical, Inc., Sunnyvale, Calif., is a minimally invasive teleoperated surgical system that offers patients many benefits, such as reduced trauma to the body, faster recovery, and shorter hospital stay. One key component of the da Vinci® Surgical System is a capability to provide two-channel (i.e., left and right) video capture and display of visible images to provide stereoscopic viewing for the surgeon.

Such electronic stereoscopic imaging systems may output high definition video images to the surgeon, and may allow features such as zoom to provide a “magnified” view that allows the surgeon to identify specific tissue types and characteristics, as well as to work with increased precision.

One problem encountered in acquiring left and right images is that the left and right images may not be aligned, e.g., one of the left and right images may displaced vertically by a number of pixels from the other of the left and right images. The misalignment is fatiguing and inhibits forming a stereoscopic image from the two images by a surgeon.

The misalignment is caused by differences in the optical paths of the left and right images prior to their acquisition. One solution to this misalignment is to place a target device on the end of the endoscope that reflects a specific pattern, such a cross. The reflected left and right visible images, which each include a cross, are acquired in the camera as left and right images.

The acquired left image is presented in a first color, e.g., a green cross, and the acquired right image is presented in a second color, e.g., a red cross, in the display viewed by the surgeon. The surgeon pushes a button to move the two crosses into alignment. The minimally invasive surgical system effectively remembers the alignment and adjusts subsequent acquired visible images so that the left and right images are properly aligned when displayed for viewing. A more detailed description of one example of this alignment process is described in U.S. Pat. No. 7,277,120 (filed Mar. 7, 2004), which is incorporated herein by reference in its entirety.

### SUMMARY

In one aspect, a minimally invasive surgical system includes an illuminator. The illuminator includes a visible color component illumination source and a hardware non-visible fluorescence emission illumination source.

In one aspect, the visible color component illumination source is included in a plurality of visible color component illumination sources. The plurality of visible color component illumination sources comprises a plurality of light emitting diodes. The plurality of light emitting diodes (LEDs) includes a red LED, two green LEDs, and a blue LED.

In another aspect, the fluorescence emission illumination has a wavelength in the near infrared spectrum of the electromagnetic radiation spectrum. In yet another aspect, the wavelength is in a range in the near infrared with a peak at 835 nm.

In still another aspect, the hardware fluorescence emission illumination source is tunable. Thus, an output wavelength of the hardware fluorescence emission illumination source can be set to a value that corresponds to an emission maximum of a selected fluorophore.

A method includes outputting target image illumination light in a first spectrum from an illumination source device. The first spectrum comprises at least a portion of the visible spectrum. The method further includes outputting target image illumination light in a second spectrum from the illumination source device. The second spectrum comprises non-visible light with a wavelength in the same range as wavelengths in an emission from a fluorophore.

### BRIEF DESCRIPTION OF THE DRAWINGS

FIG. 1 is a high level diagrammatic view of a minimally-invasive teleoperated surgical system that includes an illuminator having a visible color component illumination source and a hardware non-visible fluorescence emission illumination source.

FIGS. 2A to 2E are more detailed illustrations of an example of the illuminator.

In the drawings, the first digit of a reference number indicates the figure in which the element with that reference number first appeared.

### DETAILED DESCRIPTION

As used herein, electronic stereoscopic imaging includes the use of two imaging channels (i.e., channels for left and right images).

As used herein, a stereoscopic optical path includes two channels in an endoscope for transporting light from tissue, or from a target (i.e., channels for left and right images). The light transported in each channel represents a different view of the tissue/target. The light can include one or more images. Without loss of generality or applicability, the aspects described more completely below also could be used in the context of a field sequential stereo acquisition system and/or a field sequential display system.

As used herein, an illumination path includes a path in an endoscope providing illumination to a target, or to tissue.

As used herein, images captured in the visible electromagnetic radiation spectrum are referred to as acquired visible images.

As used herein, white light is visible white light that is made up of three (or more) visible color components, e.g., a red visible color component, a green visible color component, and a blue visible color component. If the visible color components are provided by an illuminator, the visible color components are referred to as visible color illumination components. White light may also refer to a more continuous spectrum in the visible spectrum as one might see from a heated tungsten filament, for example.

As used herein, a visible image includes a visible color component.

As used herein, a non-visible image is an image that does not include any of the three visible color components; thus, a non-visible image is an image formed by light outside the range typically considered visible.

As used herein, images captured in the visible electromagnetic radiation spectrum are referred to as acquired visible images.

As used herein, images captured as the result of fluorescence are referred to herein as acquired fluorescence images. There are various fluorescence imaging modalities. Fluorescence may result from the use of, for example, injectable dyes, fluorescent proteins, or fluorescent tagged antibodies. Fluorescence may result from, for example, excitation by laser or other energy source. Fluorescence images can provide vital in vivo patient information that is critical for surgery, such as pathology information (e.g., fluorescing tumors) or anatomic information (e.g., fluorescing tagged tendons).

As used herein, images captured as the result of illumination from a hardware non-visible fluorescence emission illumination source are referred to as artificial fluorescence images. An artificial fluorescence image is the same as a fluorescence image except the mechanism used to produce the artificial fluorescence image is different.

In a typical minimally invasive surgical field, certain tissue types are difficult to identify, or tissue of interest may be at least partially obscured by other tissue. This complicates the surgical procedure.

In some applications, fluorescence images and reflected white light images are used in minimally invasive surgery. The fluorescence images assist in identifying tissue of interest.

When fluorescence images are not in the visible spectrum, the prior art method of aligning left and right images fails to align the visual images and the fluorescence images. Non-visible images are affected differently from visible images by the optical path in the endoscope. Thus, a visible image and a non-visible fluorescence image of the same tissue may be displaced when viewed by a surgeon in stereoscopic display 151 of minimally invasive surgical system 100.

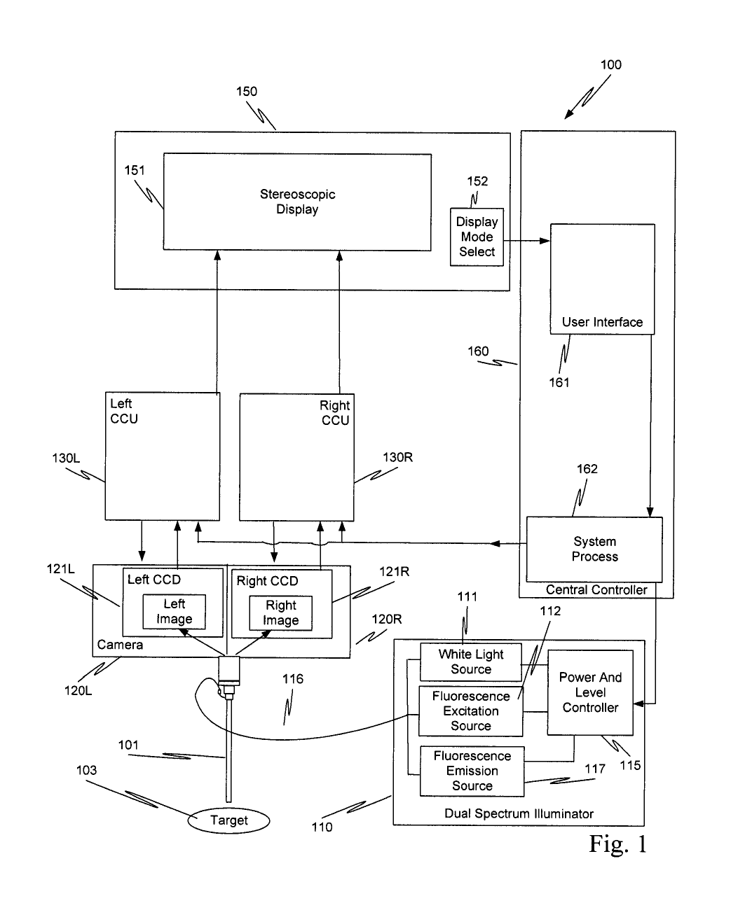

Aspects of this invention facilitate properly aligning visible and non-visible images from a surgical field that are acquired by cameras 120L, 120R (FIG. 1) in minimally invasive surgical system 100, e.g., the da Vinci® minimally invasive teleoperated surgical system commercialized by Intuitive Surgical, Inc. of Sunnyvale, Calif. In one aspect, a hardware non-visible fluorescence emission illumination source 117 provides fluorescence illumination that is not blocked by filters in the optical system. The fluorescence illumination has a wavelength that is the same as an emission wavelength of a fluorophore. Thus, hardware non-visible fluorescence emission illumination source 117 provides non-visible illumination that includes a wavelength in the same range as the wavelengths in the emission from a fluorophore. In one aspect, hardware fluorescence emission illumination source 117 is tunable so that an output wavelength of hardware fluorescence emission illumination source 117 can be set to a value that corresponds to an emission maximum of a selected fluorophore.

Herein, a hardware non-visible fluorescence emission illumination source is an illumination source that includes hardware components and can be powered off and on. The hardware non-visible fluorescence emission illumination source is defined as a hardware source to differentiate the illumination from emissions from fluorophores excited by an appropriate wavelength of light.

As explained more completely below, illumination from hardware non-visible fluorescence emission illumination source 117 can be used for a wide variety of functions in the minimally invasive surgical system 100 in addition to aligning visible and non-visible images. Hardware non-visible fluorescence emission illumination source 117 can be used in demonstrating that minimally invasive surgical system 100 is acquiring, processing, and displaying fluorescence images correctly before clinical use, e.g., can be used to verify system functionality. Hardware non-visible fluorescence emission illumination source 117 can be used in calibration of various elements within minimally invasive surgical system 100, e.g., camera control units 130L, 130R, and power and level controller 115.

In the following description, a minimally invasive surgical system 100 that includes hardware non-visible fluorescence emission illumination source 117, sometime referred to as fluorescence emission source 117, is described. System 100 and source 117 are illustrative only and are not intended to limit fluorescence emission source 117 to this specific system or configuration.

In this example, a surgeon at surgeon's console 150 remotely manipulates an endoscope 101 mounted on a teleoperated robotic manipulator arm (not shown). There are other parts, cables, etc. associated with the da Vinci® Surgical System, but these are not illustrated in FIG. 1 to avoid detracting from the disclosure. Further information regarding minimally invasive surgical systems may be found for example in U.S. patent application Ser. No. 11/762,165 (filed Jun. 13, 2007; disclosing Minimally Invasive Surgical System) and U.S. Pat. No. 6,331,181 (filed Dec. 18, 2001; disclosing Surgical Robotic Tools, Data Architecture, and Use), both of which are incorporated herein by reference.

An illumination system, e.g., dual spectrum illuminator 110, is coupled to endoscope 101. Dual spectrum illuminator 110, in one aspect, includes a white light source 111, a fluorescence excitation source 112, and fluorescence emission source 117. The on and off state of each of sources 111, 112, and 117 is independently controllable by power and level controller 115 in response to instruction from system process 162. In addition, at least the brightness of the output illumination of white light source 111 is controlled by power and level controller 115 in response to instructions from system process 162.

Typically, three visible color components make up white light, i.e., white light includes a first visible color component, a second visible color component, and a third visible color component. Each of the three visible color components is a different visible color component, e.g., a red component, a green component, and a blue component.

In one aspect, white light source 111 includes a source for each of the different visible color illumination components. For a red-green-blue implementation, in one example, the sources are light emitting diodes (LEDs)—a red LED, two green LEDs, and a blue LED. Table 1 gives the output peak wavelength for each of the LEDs used in this example.

TABLE 1

| Visible Color | |
|---|---|
| Illumination Component | Wavelength |
| Red | 620 nm |
| Green 1 | 530 nm |
| Green 2 | 512 nm |
| Blue | 460 nm |

The use of LEDs in white light source 111 is illustrative only and is not intended to be limiting. White light source 111 could also be implemented with multiple laser sources or multiple laser diodes instead of LEDs for example. Alternatively, white light source 111 could use a Xenon lamp with an elliptic back reflector and a band pass filter coating to create broadband white illumination light for visible images. The use of a Xenon lamp also is illustrative only and is not intended to be limiting. For example, a high pressure mercury arc lamp, other arc lamps, or other broadband light sources may be used.

When the fluorescence excitation wavelength occurs outside the visible spectrum (e.g., in the near infrared (NIR) spectrum), a laser module (or other energy source, such as a light-emitting diode or filtered white light) is used as fluorescence excitation source 112. When the fluorescence emission occurs outside the visible spectrum (e.g., in the near infrared (NIR) spectrum), a laser module or a laser diode (or other energy source, such as a light-emitting diode or filtered white light) is used as fluorescence emission source 117.

Thus, in one aspect, fluorescence is triggered by light from a laser module in fluorescence excitation source 112. As an example, fluorescence was excited using an 808 nm laser, and the fluorescence emission maximum was at 835 nm. For this example, fluorescence emission source 117 is a laser with an 835 nm wavelength output.

Dual spectrum illuminator 110 is used in conjunction with at least one illumination path in stereoscopic endoscope 101 to illuminate target 103, or in clinical use, tissue of a patient.

In one example, dual spectrum illuminator 110 has several modes of operation: a normal display mode; an augmented display mode; and an emission mode. In the normal display mode, white light source 111 provides illumination that illuminates target 103 in white light. Fluorescence excitation source 112 and fluorescence emission source 117 are not used in the normal display mode.

In the augmented display mode, fluorescence excitation source 112 is turned on, and fluorescence emission source 117 is turned off. Fluorescence excitation source 112 provides a fluorescence excitation illumination component that excites fluorescence of tissue. For example, narrow band light from fluorescence excitation source 112 is used to excite tissue-specific fluorophores so that fluorescence images of specific tissue within the scene are acquired by cameras 120L, 120R.

In the augmented mode, white light source 111 provides, in one aspect, one or more visible color illumination components to illuminate target 103, or in clinical use to illuminate tissue. In this aspect, both visible and fluorescence images are acquired. In another aspect, none of the visible color components of white light are used when fluorescence excitation source 112 is on.

In the emission mode of operation, white light source 111 provides, in one aspect, one or more visible color components to illuminate target 103. In another aspect, none of the visible color components of white light are used in the emission mode of operation. Fluorescence emission source 117 provides a fluorescence emission illumination that is reflected by target 103. The reflected fluorescence emission illumination is artificial fluorescence. The artificial fluorescence includes wavelengths that would be emitted by an excited fluorophore and so is the same as fluorescence.

In any of the modes of operation of dual spectrum illuminator 110, the light from the light source or light sources is directed into a fiber optic bundle 116. Fiber optic bundle 116 provides the light to an illumination path in stereoscopic endoscope 101 that in turn directs the light to target 103, or to tissue when system 100 is in clinical use.

Endoscope 101 also includes, in one aspect, two optical channels for passing light reflected from target 103. The reflected white light or a reflected visible color component is used to form a normal visible image or images. Reflected non-visible light from fluorescence emission source 117 is used to form a non-visible artificial fluorescence image that is equivalent to a non-visible fluorescence image.

The reflected light from target 103 (FIG. 1) is passed by the stereoscopic optical path in endoscope 101 to cameras 120L, 120R. In the various modes of operation, left image CCD 121L acquires a left image and right image CCD 121R acquires a right image. Each of left image CCD 121L and right image CCD 121R can be multiple CCDs that each capture a different visible color component; a single CCD with different regions of the CCD that capture a particular visible color component, etc. A three-chip CCD sensor is illustrative only. A single CMOS image sensor with a color filter array or a three-CMOS color image sensor assembly may also be used.

Camera 120L is coupled to a stereoscopic display 151 in surgeon's console 150 by a left camera control unit 130L. Camera 120R is coupled to stereoscopic display 151 in surgeon's console 150 by a right camera control unit 130R. Camera control units 130L, 130R receive signals from a system process 162. System process 162 represents the various controllers in system 100.

Display mode select switch 152 provides a signal to a user interface 161 that in turn passes the selected display mode to system process 162 in a central controller 160. Various controllers within system process 162 configure power and level controller 115 within dual spectrum illuminator 110, configure left and right camera control units 130L and 130R, and configure any other elements needed to process the acquired images so that the surgeon is presented the requested images in display 151.

In a normal viewing mode, visible images of target 103 are acquired by cameras 120L, 120R and displayed in stereoscopic display 151. In an augmented viewing mode, non-visible images, e.g., fluorescence images, are acquired by cameras 120L, 120R. The acquired non-visible images are processed, e.g., false colored using a visible color component, and presented in stereoscopic display 151. In some aspects, the augmented viewing mode may also capture visible images.

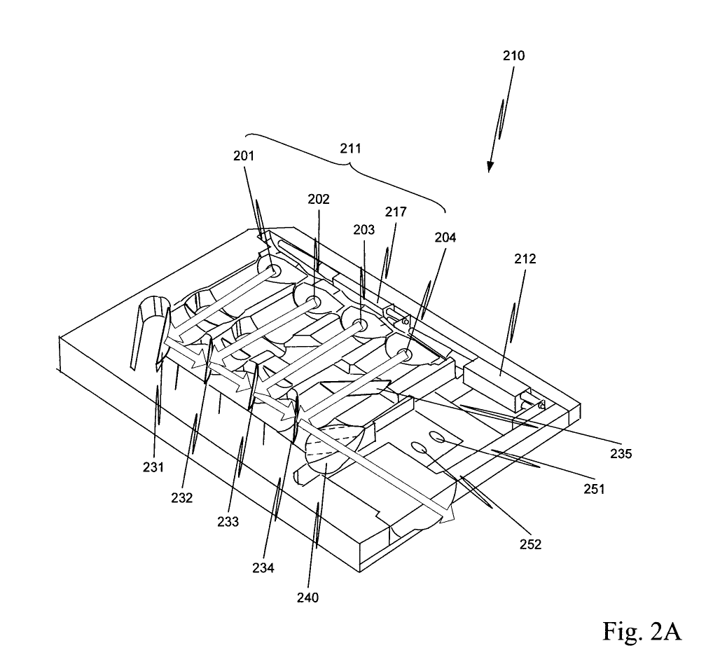

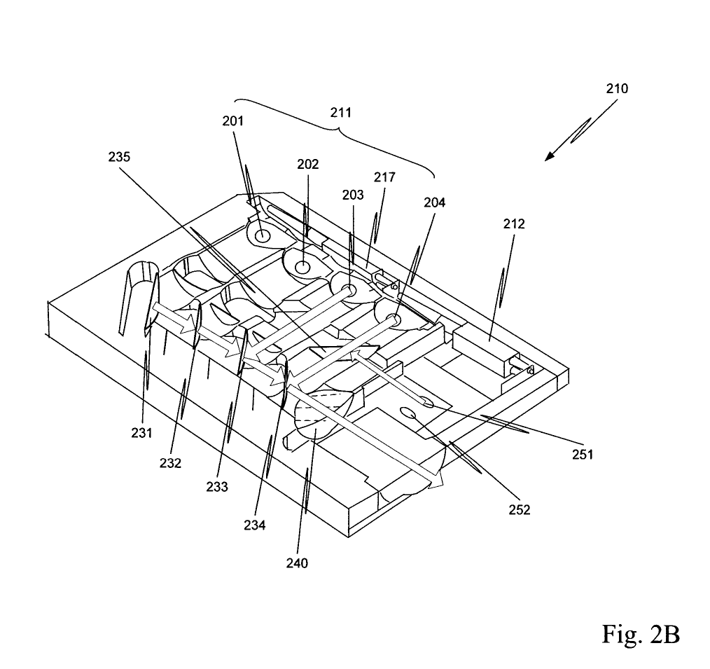

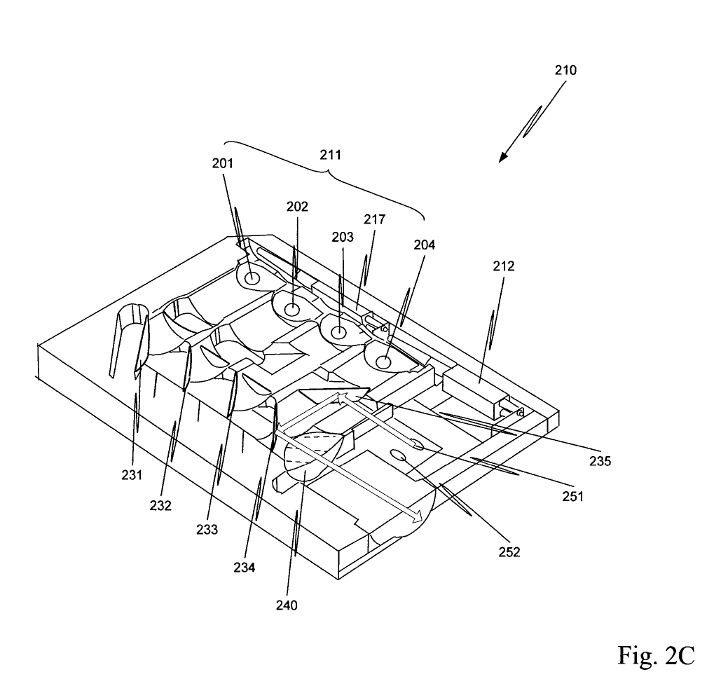

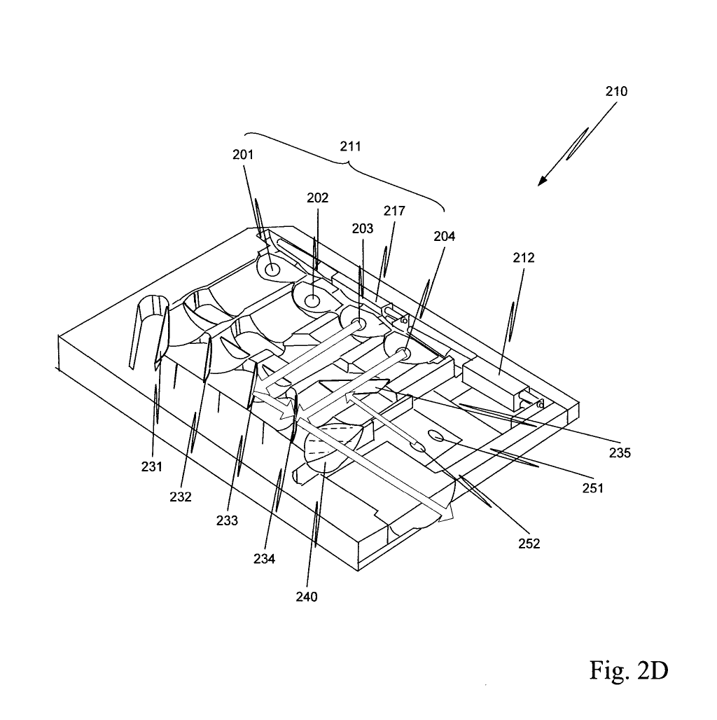

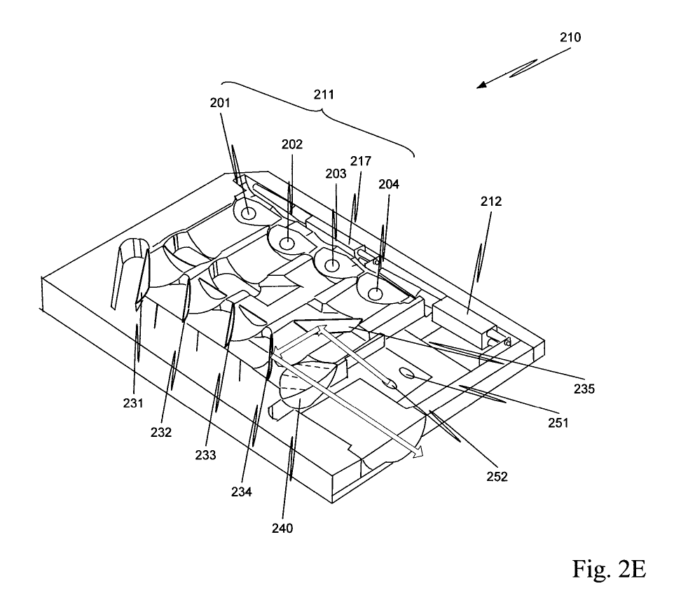

The particular technique used to combine visible images and fluorescence images for display is not essential to understanding the features of dual spectrum illuminator 110. FIG. 2A is more detailed illustration of one implementation of dual spectrum illuminator 110. Dual spectrum illuminator 210 includes a white light source 211, a fluorescence excitation source 212, and a fluorescence emission source 217.

White light source 211 includes first visible color component illumination source 201, e.g., a red LED, two second visible color component illumination sources 202, 203, e.g., two green LEDS, and a third visible color component illumination source 204, e.g., a blue LED. In one aspect, the four LEDs have the wavelengths given in TABLE 1.

In this aspect, fluorescence excitation source 212 is a near infrared laser that outputs illumination having a wavelength of 808 nm, which is illustrative of a non-visible fluorescence excitation source. Fluorescence emission source 217 is a near infrared laser that outputs illumination including an 835 nm wavelength, which is illustrative of a hardware non-visible fluorescence emission illumination source.

Light from first visible color component illumination source 201 is reflected by a mirror 231 and passes through each of dichroic mirrors 232, 233, 234. Light from a first second visible color component illumination source 202 is reflected by dichroic mirror 232 and passes through each of dichroic mirrors 233, 234. Light from a second second visible color component illumination source 203 is reflected by dichroic mirror 233 and passes through dichroic mirror 234. Light from third visible color component illumination source 204 is reflected by dichroic mirror 234.

The white light from dichroic mirror 234 passes through a lens 240 that focuses light on the end of fiber optic bundle 116.

In the configuration illustrated in FIG. 2A, fluorescence excitation source 212 and fluorescence emission source 217 are powered off and so do not emit any illumination. The configuration of dual spectrum light illuminator 210 illustrated in FIG. 2A provides only white light.

In another configuration illustrated in FIG. 2B, both visible light and non-visible fluorescence excitation light are provided to fiber optic bundle 116. Reflected visible light is a visible image of tissue. The non-visible fluorescence excitation light excites non-visible fluorescence from the tissue. The fluorescence and the fluorescence excitation light are in the near infrared spectrum of the electromagnetic radiation spectrum in this example.

Hence in the configuration of FIG. 2B, first visible color component source 201 and first second visible color component source 202 are powered off and so not provide any illumination. In one aspect, the red CCDs in cameras 120L, 120R are used to acquire left and right fluorescence images. Turning off sources 201 and 202 eliminates the possibly of reflected visible light that may affect the acquisition and display of the fluorescence images.

The operation of visible color component illumination sources 203 and 204 is the same as described above with respect to FIG. 2A. However, the illumination output levels of visible color component illumination sources 203 and 204 are reduced relative to the illumination output levels in the configuration of FIG. 2A. The illumination output level is lowered so that a proper contrast is obtained between acquired visible images and acquired fluorescence images.

In the configuration of FIG. 2B, fluorescence excitation source 212 is powered on and fluorescence emission source 217 is powered off. The output from fluorescence excitation source 212 is passed over an optical fiber to output port 251. The illumination from output port 251 is aligned with mirror 235. The non-visible fluorescence excitation illumination is reflected by mirror 235 to a portion of dichroic mirror 234 that in turn reflects the illumination to lens 240. Thus, visible light and non-visible fluorescence excitation light are provided by dual spectrum illuminator 210.

The configuration of FIG. 2C is similar to FIG. 2B, except all the visible color component illumination sources in white light source 211 are turned-off. Thus, only illumination from fluorescence excitation source 212 is provided to fiber optic bundle 116. Only non-visible fluorescence excitation light is provided by dual spectrum illuminator 210 in this configuration.

In another configuration illustrated in FIG. 2D, both visible light and non-visible fluorescence emission light are provided to fiber optic bundle 116. Reflected visible light from target 103 is acquired as a visible target image of target 103.

The non-visible fluorescence emission light illuminates target 103 also. Target 103 is configured to reflect a predetermined percentage, e.g., ten percent, of the incident non-visible fluorescence emission light. The reflected non-visible fluorescence emission light is acquired as a non-visible artificial fluorescence target image. The artificial fluorescence target image and the fluorescence emission light are both in the near infrared spectrum of the electromagnetic radiation spectrum in this example.

Thus, the illumination from dual spectrum illuminator 210 in this configuration generates a visible target image and a non-visible artificial fluorescence target image. Prior to considering the use of these target images in further detail, the configuration in FIG. 2D is described more completely.

In the configuration of FIG. 2D, first visible color component illumination source 201 and first second visible color component illumination source 202 are powered off and so not provide any illumination. This is because the red CCDs in camera 120L, 120R are used to acquire left and right artificial fluorescence target images and turning off sources 201 and 202 eliminates the possibly of reflected visible light that may affect the acquisition and display of the artificial fluorescence target images.

The operation of sources 203 and 204 is the same as described above with respect to FIG. 2A. However, the illumination output level of sources 203 and 204 is reduced relative to the illumination output level in the configuration of FIG. 2A. The illumination output level is lowered so that a proper contrast is obtained between acquired visible target images and acquired artificial fluorescence target images.

In the configuration of FIG. 2D, fluorescence emission source 217 is powered on. Fluorescence excitation source 212 is not powered on. The output from fluorescence emission source 217 is passed over an optical fiber to output port 252. While the illumination from output port 251 is aligned with mirror 235, illumination from output port 252 is not properly aligned with mirror 235 in this implementation. However, the efficiency of folding the non-visible fluorescence emission illumination into the beam provided to fiber optic bundle 116 is not critical and so exact alignment is not required.

The non-visible fluorescence emission illumination is reflected by mirror 235 to a portion of dichroic mirror 234 that in turn reflects the illumination into lens 240. Thus, visible light and non-visible fluorescence emission light are provided by dual spectrum illuminator 210.

Thus, the configuration in FIG. 2D is an example of an illuminator that includes a visible color component illumination source and a hardware non-visible fluorescence emission illumination source. The illuminator outputs target image illumination light in a first spectrum, where the first spectrum is a portion of the visible spectrum. The illuminator also outputs target image illumination light in a second spectrum, where the second spectrum includes non-visible light with a wavelength in the same range as the wavelengths in the emission from a fluorophore.

As indicated above, the visible light and the non-visible fluorescence emission light are reflected by target 103 as visible and non-visible light. Thus, left and right visible target images and left and right non-visible artificial fluorescence target images are acquired by cameras 120L, 120R.

In one example, the target is a camera alignment target, and the acquired target images are camera alignment target images. The left and right visual camera alignment target images are displayed in stereoscopic display so that the user can align the images. See for example, U.S. Pat. No. 7,277,120, which was previously incorporated herein by reference. Next, the aligned visual target images and the artificial fluorescence target images are presented in the stereoscopic display. Again, the visual and fluorescence target images are aligned by the user.

In another aspect, the visual images and the artificial fluorescence target images are superimposed and presented in display 151. As indicated above, the illumination output of any active visual color component illumination source is reduced when visual and artificial fluorescence target images are acquired together. Thus, the brightness of the visual color illumination components can be systematically adjusted and visual and artificial fluorescence target images acquired until the contrast between the displayed artificial fluorescence and visual target images reaches a desired level.

In some aspects of calibrating and establishing the functionally of system 100, only the artificial fluorescence target images may be needed. In the configuration of FIG. 2E, fluorescence excitation source 212 and all the sources in white light source 211 are turned-off. Thus, only illumination from fluorescence emission source 217 is provided to fiber optic bundle 116. Thus, only non-visible fluorescence emission light is provided by dual spectrum illuminator 210 in this configuration.

The non-visible fluorescence emission light illuminates target 103, which reflects light that is acquired as an artificial fluorescence target image. System 100 is calibrated and aligned so that the artificial fluorescence target image is displayed on display 151. For example, gains are adjusted as needed. This assures that when system 100 is used in a clinical setting, if fluorescence is generated within the field of view of endoscope 101, system 100 captures and processes that fluorescence correctly, i.e., if fluorescence is there, system 100 sees the fluorescence.

In one aspect, hardware fluorescence emission illumination source 217 is tunable so that an output wavelength of hardware fluorescence emission illumination source 217 can be set to a value that corresponds to an emission maximum of a selected fluorophore. In some aspects, dichroic mirror 234 includes a plurality of coatings and is slidable. Thus, as the output of source 217 is changed, mirror 234 is automatically positioned so that mirror reflects the illumination from source 217.

In the above examples, a stereoscopic endoscope was used. This is illustrative only and is not intended to be limiting. The features described are directly applicable to a monoscopic endoscope used to capture fluorescence images.

In addition, while the above examples described the fluorescence emission source as being included with a dual spectrum illuminator, this also is illustrative only. For example, the fluorescence emission source could be included in target 103 so that fluorescence viewed by system 100 is a direct emission from target 103, i.e., a direct emission from the source. In one aspect, the source is a hardware source such as an LED or a laser diode. In another aspect, the source is a fluorophore that is excited by the fluorescence excitation source. Alternatively, the hardware fluorescence emission source could be included in target 103 so that the fluorescence viewed by system 100 is light from the hardware source that is reflected by target 103.

The above description and the accompanying drawings that illustrate aspects and embodiments of the present inventions should not be taken as limiting—the claims define the protected inventions. Various mechanical, compositional, structural, electrical, and operational changes may be made without departing from the spirit and scope of this description and the claims. In some instances, well-known circuits, structures, and techniques have not been shown or described in detail to avoid obscuring the invention.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the device in use or operation in addition to the position and orientation shown in the figures. For example, if the device in the figures is turned over, elements described as “below” or “beneath” other elements or features would then be “above” or “over” the other elements or features. Thus, the exemplary term “below” can encompass both positions and orientations of above and below. The device may be otherwise oriented (rotated 90 degrees or at other orientations) and the spatially relative descriptors used herein interpreted accordingly. Likewise, descriptions of movement along and around various axes include various special device positions and orientations.

The singular forms “a”, “an”, and “the” are intended to include the plural forms as well, unless the context indicates otherwise. The terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

All examples and illustrative references are non-limiting and should not be used to limit the claims to specific implementations and embodiments described herein and their equivalents. The headings are solely for formatting and should not be used to limit the subject matter in any way, because text under one heading may cross reference or apply to text under one or more headings. Finally, in view of this disclosure, particular features described in relation to one aspect or embodiment may be applied to other disclosed aspects or embodiments of the invention, even though not specifically shown in the drawings or described in the text.

We claim:

1. A minimally invasive surgical system comprising:
an illuminator,
wherein the illuminator comprises a visible color component illumination source and a hardware non-visible fluorescence emission illumination source.

2. The minimally invasive surgical system of claim 1, wherein the visible color component illumination source comprises a light emitting diode.

3. The minimally invasive surgical system of claim 1, wherein the visible color component illumination source is included in a plurality of visible color component illumination sources.

4. The minimally invasive surgical system of claim 3, the plurality of visible color component illumination sources comprises a plurality of light emitting diodes.

5. The minimally invasive surgical system of claim 1, wherein the visible color component illumination source comprises a laser diode.

6. The minimally invasive surgical system of claim 1, wherein the visible color component illumination source comprises a laser.

7. The minimally invasive surgical system claim 1, wherein the fluorescence emission illumination has a wavelength in the near infrared spectrum.

8. The minimally invasive surgical system of claim 7, wherein the wavelength is about 835 nm.

9. The minimally invasive surgical system claim 1, wherein the hardware fluorescence emission illumination source is tunable so that an output wavelength of the hardware fluorescence emission illumination source can be set to a value that corresponds to a emission maximum of a selected fluorophore.

10. The surgical system of claim 1, further comprising a fluorescence excitation illumination source.

11. The surgical system of claim 10, wherein the fluorescence excitation illumination source comprises a laser diode.

12. The surgical system of claim 10, wherein the fluorescence excitation illumination source comprises a fiber coupled laser diode.

13. A method comprising:
outputting target image illumination light in a first spectrum from an illumination source device, wherein the first spectrum comprises at least a portion of the visible spectrum; and
outputting target image illumination light in a second spectrum from the illumination source device, wherein the second spectrum comprises non-visible light including a wavelength in a range of emission wavelengths from a fluorophore.

14. The method of claim 13 further comprising:
illuminating a target with the output target image illumination light in the first and second spectrums,
wherein the target reflects the output target image illumination light in the first and second spectrums and the reflected light is acquired as first and second target images.

15. The method of claim 14, wherein the first and second target images comprise images of a minimally invasive surgical camera alignment target.

16. The method of claim 13, where the outputting target image illumination light in a second spectrum comprises outputting near infrared target image illumination.
